(19) United States
(12) Patent Application Publication
(10) Pub. No.: US 2014/0249566 A1
(43) Pub. Date: Sep. 4, 2014

(54) EMBOLIC PROTECTION SHIELD

(71) Applicant: AGA MEDICAL CORPORATION, Plymouth, MN (US)

(72) Inventors: Chris Quinn, Minneapolis, MN (US); Amy Rochelle Raatikka, Plymouth, MN (US)

(73) Assignee: AGA MEDICAL CORPORATION, Plymouth, MN (US)

(21) Appl. No.: 13/782,677

(22) Filed: Mar. 1, 2013

(51) Int. Cl. A61F 2/01

(52) U.S. Cl. CPC A61F 2/013
USPC 606/200

(57) ABSTRACT

A vessel protector for capturing or filtering material in the aortic arch includes at least one shield formed in a planar or three-dimensional shape. The shield includes a body formed from a filtering material and may be formed of a shape memory material. The shield may alternate between a collapsed configuration for delivery and an expanded configuration during use. A catheter may be used to deliver the vessel protector into the aortic arch.

### BACKGROUND OF THE INVENTION

The present invention is related to protecting against embolism, and more particularly to devices, systems, and methods for the filtration of debris within blood vessels.

A frequent risk in medical procedures is the potential dislodging of damaging debris such as atherosclerosis plaque and/or calcified tissue in the patient's bloodstream. Such debris may take the form of emboli, which may travel through the patient's vasculature and become lodged in a position that blocks blood flow. For example, during coronary interventions, emboli may become dislodged and migrate to the carotid arteries, possibly blocking the carotid arteries and causing a stroke.

### BRIEF SUMMARY OF THE INVENTION

In accordance with the device, system and method, several examples of vessel protectors are provided. Specifically, shields are employed to protect vessels emanating from the aortic arch, primarily the brachiocephalic artery, the left common carotid artery, and/or possibly the left subclavian artery.

In some embodiments, a vessel protector for use with a pigtail catheter includes a pigtail catheter, an outer sheath, and a shield disposed within the outer sheath. The shield has a body formed from a filtering material and may be capable of receiving the pigtail catheter.

In some embodiments, a vessel protector includes an outer sheath, an inner shaft disposed within the outer sheath and moveable relative to the outer sheath, and a plurality of shields coupled to the inner shaft. Each of the plurality of shields has a body formed from a filtering material and the shields have a collapsed configuration and an expanded configuration. The plurality of shields may be capable of alternating between the collapsed configuration and the expanded configuration by movement of the inner shaft relative to the outer sheath.

In some embodiments, a vessel protector includes a frame including a shaft and a plurality of arched ribs connected to the shaft. The frame is formed of a shape-memory material that can be collapsed within a delivery catheter and returned to its expanded relaxed state when deployed from the delivery catheter. A plurality of shields is disposed between the plurality of arched ribs. Each of the plurality of shields has a body formed from a filtering material. The frame may be capable of collapsing to fit within a delivery catheter.

In some embodiments, a vessel protector includes a shaft having a first end and second end, and at least one shield coupled to the first end of the shaft. The at least one shield has a body formed from a filtering material. The at least one shield may be capable of collapsing to fit within a delivery catheter. The body of the at least one shield may have an expanded shape of an awning and a number of longitudinal pleats to aid in collapsing the body. The at least one shield may include a plurality of leaflets formed of a shape-memory material that can be collapsed within a delivery catheter and returned to a radially expanded relaxed state when deployed from the delivery catheter.

In some embodiments, a method for protecting blood vessels during a medical procedure includes inserting a vessel protector device into a patient's body. The vessel protector device including an outer sheath, an inner shaft disposed within the outer sheath and moveable in a longitudinal direction relative to the outer sheath, and at least one shield coupled to the inner shaft at a first end of the shield and to outer sheath at a second end of the shield. Each of the at least one shield has a body formed from a filtering material, and the body has a collapsed configuration and an expanded configuration. The method further includes positioning the vessel protector device adjacent an open end of at least one blood vessel and moving the outer sheath relative to the inner shaft to place the body of the at least one shield in the expanded configuration to filter blood passing through the body into the at least one blood vessel.

### BRIEF DESCRIPTION OF THE DRAWINGS

Various embodiments of the present system and method will now be discussed with reference to the appended drawings. It is appreciated that these drawings depict only some embodiments and are therefore not to be considered as limiting the scope of the present system and method.

FIG. 1 is a schematic illustration of an aorta;

FIG. 2 is a side perspective view of a shield in accordance with the first embodiment;

FIG. 3 is a side perspective view of a shield having a marker band in accordance with another embodiment;

FIG. 4A is a side perspective view of a vessel protector device for use with a pigtail catheter in accordance with a second embodiment;

FIG. 4B is a schematic cross-sectional view of the vessel protector device of FIG. 4A along line A-A;

FIG. 5A is a side perspective view of a tube for forming the shield of the vessel protector device of FIG. 4A;

FIG. 5B is a side perspective view of the tube of FIG. 5A folded over itself to form a double-layer tube;

FIG. 5C is a side perspective view of the tube of FIG. 5A after being collapsed to form a C-shaped shield;

FIGS. 5D-F are schematic cross-sectional views of variations of a shield for use with the vessel protector device of FIG. 4A;

FIG. 6A is schematic illustration of the use of the vessel protector device of FIG. 4A in the aorta;

FIG. 6B is schematic illustration of the vessel protector device of FIG. 4A in its expanded condition in the aorta;

FIG. 7A is a side perspective view of a vessel protector device in accordance with a third embodiment in a collapsed condition;

FIG. 7B is a side perspective view of the vessel protector device of FIG. 7A in an expanded condition;

FIG. 7C is a bottom view of a shield of the vessel protector device of FIG. 7A in an expanded condition;

FIG. 7D is schematic illustration of the use of the vessel protector device of FIG. 7A in the aorta;

FIG. 8A is a side perspective view of a vessel protector device in accordance with a fourth embodiment in a collapsed condition;

FIG. 8B is a top view of a shield of the vessel protector device of FIG. 8A in the collapsed condition;

FIG. 8C is a side perspective view of the vessel protector device of FIG. 8A in an expanded condition;

FIG. 8D is a top view of the shield of the vessel protector device of FIG. 8A in an expanded condition;

FIG. 8E is schematic illustration of the use of the vessel protector device of FIG. 8A in the aorta;

FIG. 9A is a side perspective view of a vessel protector device in accordance with a fifth embodiment in an expanded condition;

FIG. 9B is a side perspective view of the vessel protector device of FIG. 9A in a first collapsed condition;

FIG. 9C is a side perspective view of the vessel protector device of FIG. 9A in an alternative collapsed condition;

FIG. 10A is a side perspective view of a vessel protector device in accordance with a sixth embodiment in an expanded condition;

FIG. 10B is a side perspective view of the vessel protector device of FIG. 10A in a collapsed condition;

FIG. 11A is a side perspective view of a vessel protector device in accordance with a seventh embodiment in an expanded condition; and

FIG. 11B is a side perspective view of the vessel protector device of FIG. 11A in a collapsed condition.

### DETAILED DESCRIPTION

In the description that follows, the terms “proximal” and “distal” are to be taken as relative to a user (e.g., a surgeon or an interventional cardiologist) of the disclosed devices and methods. Accordingly, “proximal” is to be understood as relatively close to the user, and “distal” is to be understood as relatively farther away from the user.

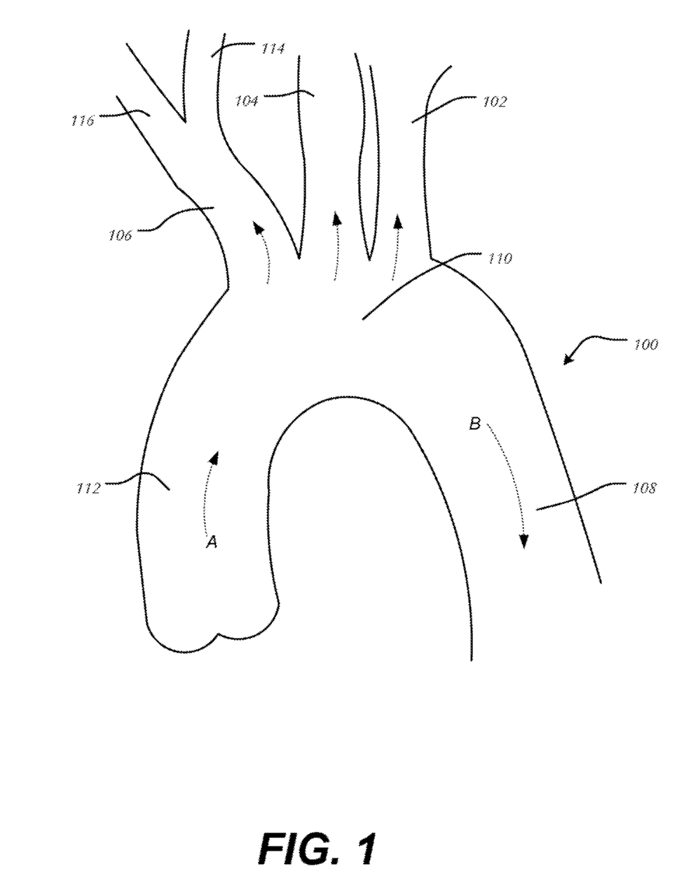

FIG. 1 illustrates the aorta 100, the largest artery in the body, originating from the left ventricle (not shown) and extending down to the abdomen. Blood flows as indicated by arrow “A” from the left ventricle, through the aortic valve (not shown), through the ascending aorta 112 to the aortic arch 110. Branching from aortic arch 110 are commonly three major arteries: brachiocephalic artery 106, which supplies blood to the right arm and the head and neck, left common carotid artery 104, which supplies blood to the head and neck, and left subclavian artery 102, which supplies blood to the left arm. Branching off brachiocephalic artery 106 are right subclavian artery 116 (supplying blood to the right arm) and right common carotid artery 114 (supply blood to the head and neck). Variations may occur in the number and position of vessels arising from the aortic arch. For example, it has been found that in certain instances, the brachiocephalic and left common carotid unite to form one branch. Blood from ascending aorta 112 not passing through one of these three branch arteries continues down the descending aorta 108 as shown by arrow “B”.

The risk of stroke associated with medical procedures may be reduced by using a filter to protect those vessels which are at risk from the procedure. Specifically, shields deployed in the aortic arch or any one of the aforementioned branches may be useful to protect the vessels from liberated emboli.

FIG. 2 is a side perspective view of shield 230 in accordance with one embodiment of the present disclosure. While FIG. 2 illustrates a single shield 230, it will be understood that a vessel protector may include two, three, four or more shields each having a body.

Shield 230 may include a planar or three-dimensional body 235 extending between leading end 234 and trailing end 232. Body 235 may be formed from a woven, braided, or knitted material having openings of sufficient size to allow the passage of blood, but block the passage of particulates greater than a certain size. As such, the material of body 235 acts as a filter. Body 235 may also have an expanded cylindrical cross-sectional shape in use, but may be collapsible to a smaller width such as by stretching to fit within a catheter for delivery into and removal from the patient as will be described below. In this regard, body 235 may be formed from a shape-memory material, such as nickel titanium alloy (NiTi, or “nitinol”), that is readily collapsible and that will automatically expand to an operative shape upon deployment. For example body 235 may be formed from braided nitinol wire, from nitinol wire woven to form a mesh, from a nitinol tube perforated with a plurality of small apertures, and other such structures.

Alternatively, body 235 may be formed from other metals, metal alloys, or polymers such as nylon or polyethylene, that are capable of being woven or otherwise formed into a porous shaped body that may be collapsed and fully or partially disposed within a catheter for delivery into and removal from the patient, but that will return to its expanded shape when deployed from the sheath. Still further, body 235 may be formed with a nitinol or other shape-memory frame supporting a fabric layer formed from woven polyester, nylon, polyethylene or similar material.

As noted above, the material forming body 235 should have openings of sufficient size to permit the passage of blood, but block the passage of particulates greater than a certain size. In this regard, body 235 may include a mesh having openings between about 80 um and about 300 um. Body 235 may be self-expanding upon release from a sheath, or may require the use of one or more instruments to expand following release. Body 235, which is self-expanding, may be formed from a biocompatible elastic, superelastic, elastomeric, or shape-memory material which returns to an initial undeformed shape upon release from a catheter. Alternatively, body 235 which is not self-expanding may be formed from a biocompatible material which deforms plastically, and may employ additional snares or other devices to effect radial expansion.

In some embodiments, the weave, braid, or knit of body 235 may be varied such that the openings in the mesh vary according to their position on the body. For example, a braided body may be formed with varying opening sizes such that intermediate section 237, generally midway between the ends of body 235, has smaller mesh openings than the sections bordering leading end 234 and trailing end 232 of body 235. Body 235 with varying openings can provide finer filtering at its middle area as compared to its end areas. Other variations in opening size along the length of body 235 are also contemplated herein. Body 235 may be formed from a single layer of material. Alternatively, body 235 may be formed as a double layer of material by folding the tubular body over itself along its length. The overlapping layers effectively provide small sized openings to capture debris within the blood by providing finer filtering.

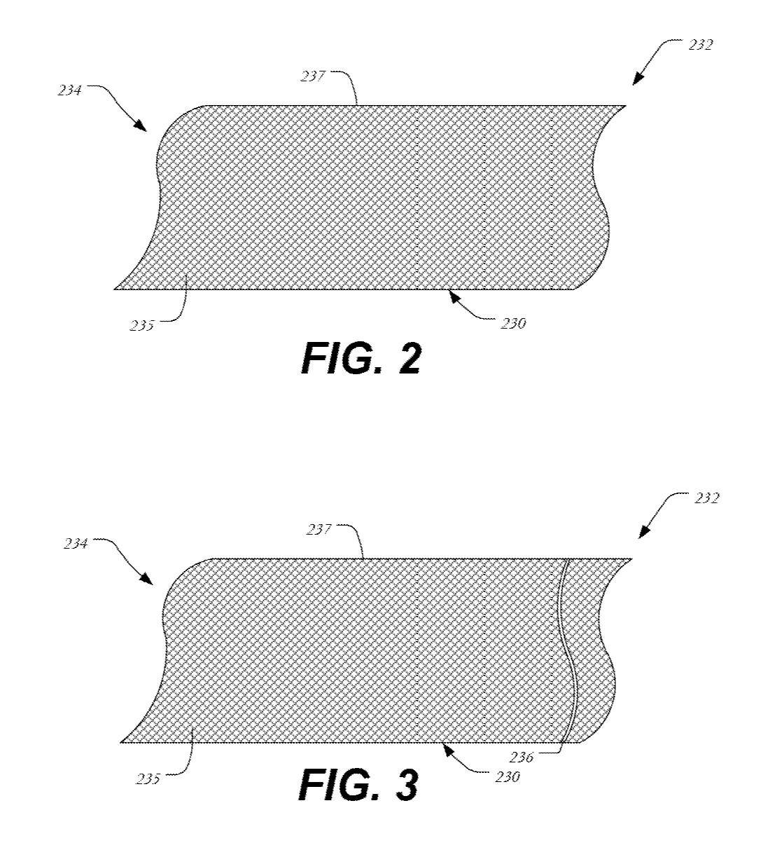

As shown in FIG. 3, body 235 may include one or more marker bands 236, disposed on the body at leading end 234, trailing end 232 or therebetween. Marker bands 236 may be radiopaque to allow for visualization of shield 230 within the patient during use. Marker bands 236 may also serve as points of attachment of shield 230 to the remaining elements of a vessel protector device as will be described below.

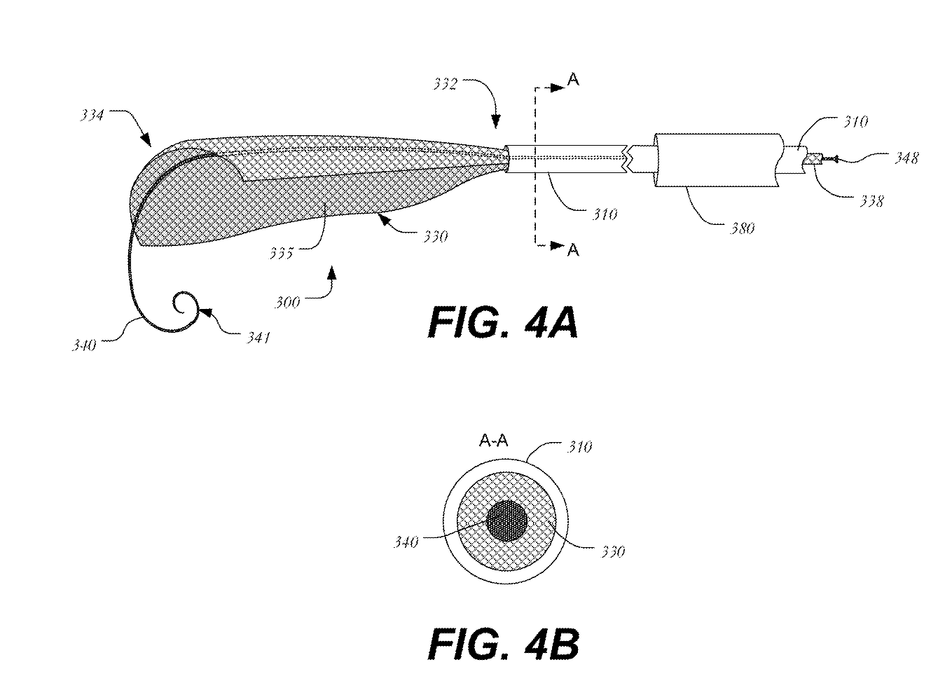

FIG. 4A is a side perspective view of vessel protector device 300 to be used in conjunction with a pigtail catheter. A conventional angiographic pigtail catheter, typically used for delivering contrast media, ends in a tightly curled tip that resembles the tail of a pig. The coiled end acts to hold the pigtail catheter in place (i.e., anchor it), and it can also be used to slow the flow of fluids injected through the catheter so that they do not burst out in a jet and cause injuries or obscure a medical imaging study. In the disclosed embodiment pigtail catheter includes elongated pigtail catheter 340 disposed within introducer catheter 380 (FIGS. 4A and 4B).

Pigtail catheter 340 may extend from beyond a distal end of the device to proximal hub 348. The distal end of pigtail catheter 340 may terminate in a tightly curled portion 341. When used in conjunction with protector device 300, pigtail catheter 340 may facilitate rotational positioning and stability of protector device 300. Introducer catheter 380 typically extends from the proximal end of the device to a location prior to the distal end of the device.

The vessel protecting component of device 300 includes a shield 330, which may extend through outer sheath 310 and attach at its proximal end to outer sheath 310 and shield hub 338. Sheath 310 may be sized according to the vessel in which it will be used. For example, when the sheath 310 is to be used in an aorta, the sheath may be sized in the range of 5 Fr to 12 Fr, depending on the aortic diameter. Shield 330 and outer sheath 310 may be disposed over pigtail catheter 340 and within introducer catheter 380, effectively being sandwiched in between the two components of pigtail catheter 340. FIG. 4B is a schematic cross-sectional view of vessel protector device 300 along line A-A of FIG. 4A. As shown, shield 330 is nested between sheath 310 and pigtail catheter 340.

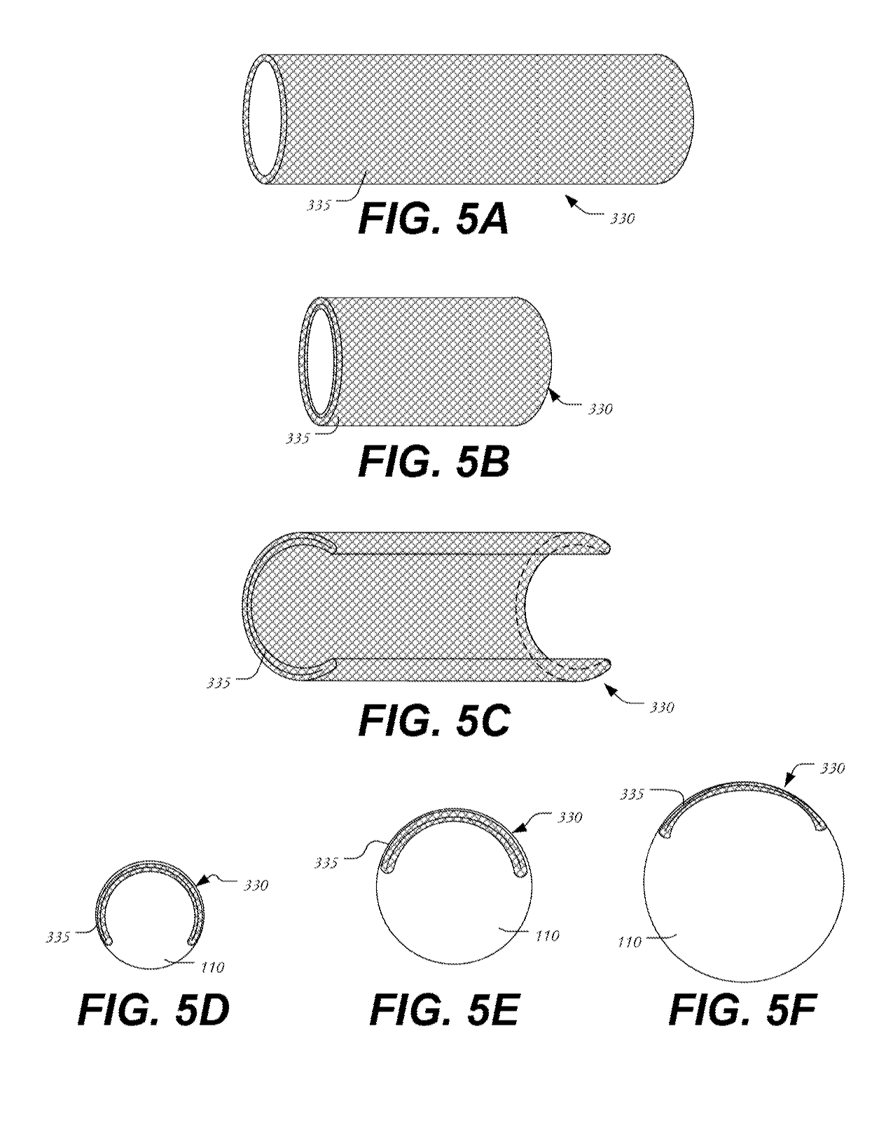

Shield 330 may include curved body 335 formed of a single or multiple layer material having leading end 334 and trailing end 332 and may be formed of any of the materials and include any of the mesh arrangements discussed above with reference to the embodiment described with reference to FIGS. 2 and 3. FIGS. 5A-F illustrate various embodiments of shield 330. It will be understood that any of the shield configurations described in the present disclosure may be combined with any of the various embodiments being presented herein. As seen in FIG. 5A, shield 330 may begin as a single-layer tube. Alternatively, a tube may be folded once over itself (or everted) to create a double-layer tube-within-tube configuration as shown in FIG. 5B. Regardless of whether a single layer or double-layer configuration is used, one wall of the tube may be collapsed into an opposing wall to form C-shaped shield 330 as shown in FIG. 5C.

FIGS. 5D-F are schematic cross-sectional views of shield 330 in use within differently-sized aortic arches 110. As shown in these figures, C-shaped shield 330 may cover different portions of the circular representation of the aortic arch depending on the size of the anatomy. In an aortic arch of small diameter (FIG. 5D), shield 330 may cover most of the circumference of the aortic arch, while in larger aortic arches, the same shield may be used to cover a smaller portion of the circumference of the aortic arch (FIGS. 5E and 5F).

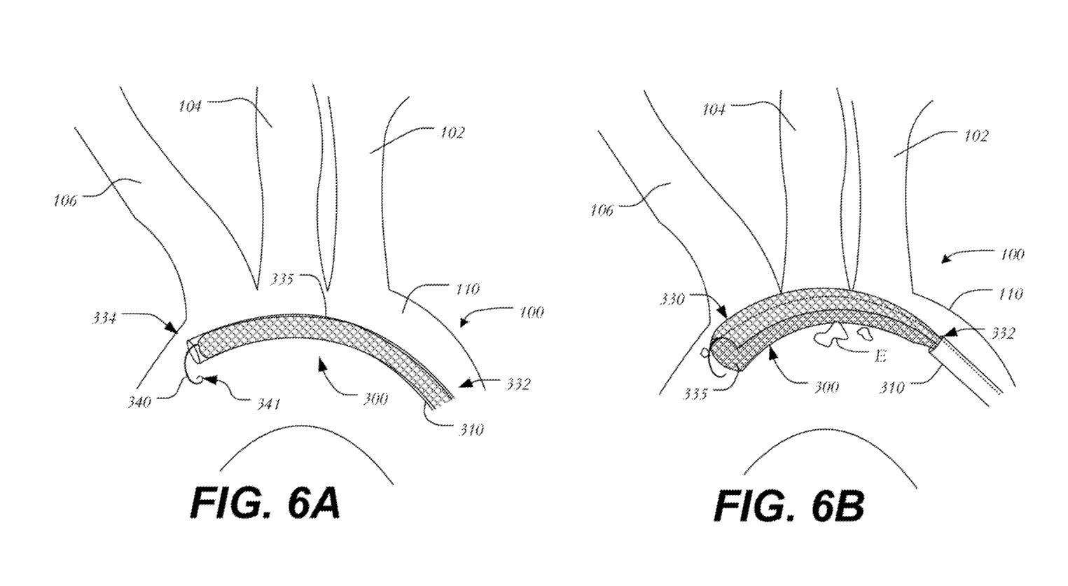

FIGS. 6A and 6B show vessel protector 300 of FIGS. 4A-B as used in a transcatheter procedure, such as for example a transcatheter aortic valve implantation (TAVI)(also known as a transcatheter aortic valve replacement (TAVR) procedure). The figure includes representations of a patient's aorta 100, left subclavian artery 102, left common carotid artery 104 and brachiocephalic artery 106, as described above with reference to FIG. 1. Vessel protector 300 has also been introduced to aorta 100 through, for example, a transfemoral approach.

An operative catheter (not shown) may be capable of delivery of a drug or device, or other therapeutic operation to or through aorta 100 via a transfemoral approach. The operative catheter may be introduced into the aortic arch 110 through the same or different approach as vessel protector 300. For example, vessel protector 300 may be introduced transfemorally while the operative catheter is introduced transapically or vice versa.

Vessel protector 300 is introduced to aorta 100 in a collapsed configuration within a delivery catheter 380 (shown in FIG. 4A) and sheath 310 as shown in FIG. 6A. Delivery catheter 380 is maneuvered until sheath 310 is in aorta 100 and in position to cover one or more of the aortic arch side branches (e.g., brachiocephalic 106, left common carotid 104, and/or left subclavian arteries 102). In this example, delivery catheter 380 terminates prior to aorta 100 and outer sheath 310 having shield 330 extends out into the aorta. Once delivery catheter 380 and sheath 310 are properly positioned, the user pulls sheath 310 proximally relative to vessel protector device 300 to retract it, thereby exposing shield 330. In some examples, contrast media may be delivered via pigtail catheter 340 through tightly curled portion 341 to aid in visualization prior to, during or after unsheathing shield 330.

Shield 330 may be formed from shape-memory material which self-expands to its original size and shape upon deployment from sheath 310. As seen in FIG. 6B, with shield 330 fully released from within sheath 310 and in proper position, body 335 covers the openings to certain arteries as desired while allowing an operative catheter to perform its intended function. That is, body 335 expands into an expanded shape from leading end 334 to trailing end 332 and acts to filter blood passing through its wall to one or more arteries.

In the scenario of FIGS. 6A and 6B, shield 330 is positioned to protect the left subclavian artery 102, left common carotid artery 104 and the brachiocephalic artery 106 from emboli that may be released during the cardiovascular procedure. That is, shield 330 covers the openings of the arteries, with the openings in the filtering material of the body permitting the passage of blood while blocking the passage of emboli (e.g. plaque and/or calcification).

It should be noted that FIGS. 6A and 6B depict an illustrative application of protector 300, and that application of the protector is not limited to the context of FIGS. 6A and 6B. For example, protector 300 may be delivered to the patient's aorta 100 through the left radial artery, left brachial artery, or left subclavian artery. Moreover, protector 300 may be used to protect vessels other than the left common carotid and brachiocephalic arteries, and may be employed in other procedures. Thus, protector 300 may be used in any procedure in which there is a possibility that plaque, emboli or other debris may be introduced into the bloodstream, and in which the protector may be positioned to capture same.

FIG. 6B illustrates the vessel protector 300 in vivo in its expanded condition. During the therapeutic operation, emboli “E” are located in the aorta. Emboli “E” may be any detached, traveling intravascular mass carried by circulation and capable of clogging arterial capillary beds. It would be beneficial to shield emboli “E” from traveling through the three upper arteries so that it does not clog a capillary bed during or after completion of the operation. Due to the size of the openings of shield 330, blood is able to flow freely through left subclavian artery 102, left common carotid artery 104 and the brachiocephalic artery 106 thereby providing sufficient blood flow to the brain, while emboli “E” becomes shielded, lodged within, and/or captured by the openings of shield 330.

As a final step, sheath 310 may be distally translated, while holding hub 348 (FIG. 4A) at a fixed distance to collapse shield 330, and delivery catheter 380 may be distally advanced over a portion of sheath 310 to fully encapsulate vessel protector device 300 as well as any entrapped emboli “E”. Once shield 330 is fully retracted within sheath 310, vessel protector 300 may be removed from the patient including any emboli that is lodged within the openings of shield 330. Any captured material will be removed from the patient along with protector 300, and thus will not present a threat of embolism.

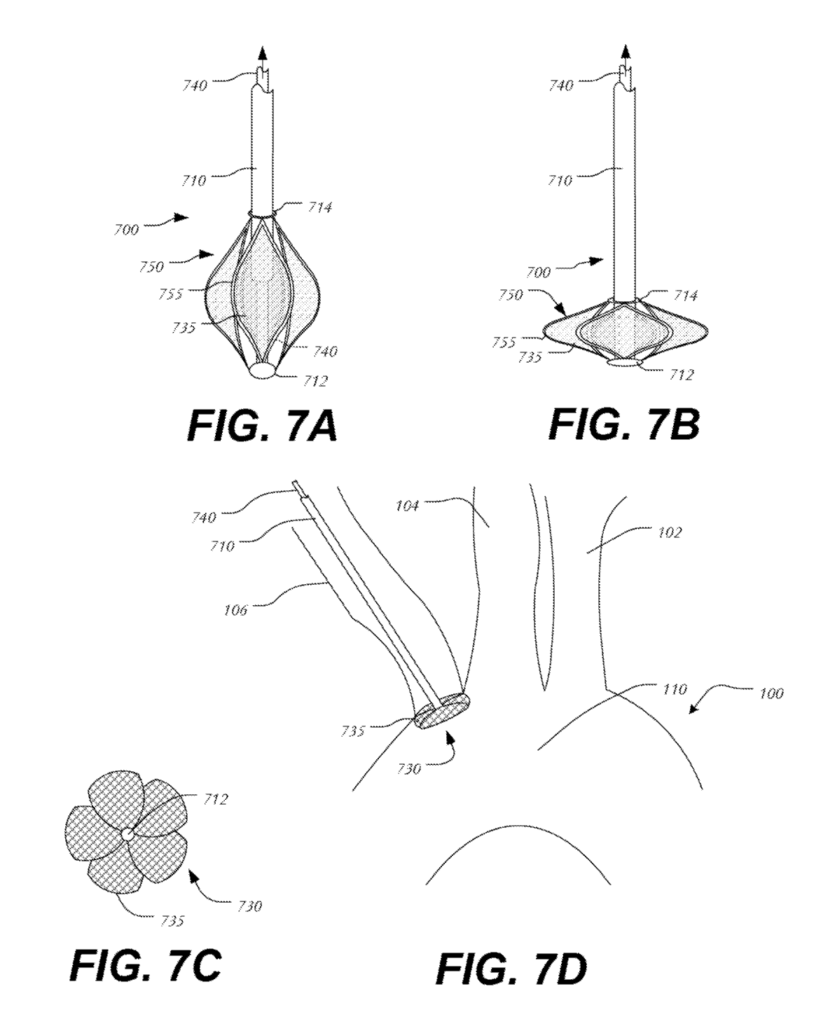

FIG. 7A is a side perspective view of vessel protector device 700 in accordance with another embodiment shown in a collapsed condition. As shown in this contracted condition, vessel protector device 700 includes elongated outer sheath 710 and inner shaft 740 coaxially disposed within outer sheath 710. Sheath 710 and inner shaft 740 are capable of longitudinal translation with respect to one another. Sheath 710 may be sized according to the vessel in which it will be used, or through which it will traverse, and may be deployed within a delivery catheter.

Vessel protector device 700 further includes deformable basket 750 coupled to sheath 710 and formed of a plurality of spaced apart flexible struts 755 extending between and connected to first joint 712 and second joint 714. First joint 712 may be connected to a distal end of inner shaft 740 while second joint 714 may be connected to sheath 710 adjacent its distal end. A number of deformable petals 735 may be connected between a pair of adjacent struts 755.

Petals 735 may be formed from a woven, braided, or knitted material having openings of sufficient size to allow the passage of blood, but block the passage of particulates greater than a certain size. As such, the material of petals 735 acts as a filter. Each petal 735 may be formed of any of the materials described above with reference to body 235 and may be configured in the same manner as body 235 such as for example, to include openings of varying sizes. Each petal 735 may resemble an eye-shape in the stretched configuration as shown in FIG. 7A that is deformable such that together the petals form a flower-shaped shield as seen in FIG. 7C.

FIG. 7B is a side perspective view of the vessel protector device 700 of FIG. 7A in an expanded condition. Expansion of vessel protector device 700 may be accomplished by proximally pulling shaft 740 toward the user while holding sheath 710 in place. Because basket 750 is connected to the distal end of shaft 740 at first joint 712 and to sheath 710 at second joint 714, pulling shaft 740 toward the user while holding sheath 710 in place serves to vertically collapse basket 750 along its longitudinal axis and radially expand struts 755. As a result, petals 735 assume an expanded configuration to form shield 730 as shown in FIG. 7C. Shield 730 includes a plurality of partially overlapping petals 735 that collectively form a filtering barrier that can be positioned across a cross-section of an artery.

FIG. 7D is schematic illustration of the use of the vessel protector device of FIG. 7A in the aorta. In one example, shown in this figure, vessel protector device 700 may be introduced toward aorta 100 in the collapsed configuration of FIG. 7A through a radial approach until the device is disposed in brachiocephalic artery 106. Once protector device 700 is properly positioned near brachiocephalic artery 106, the user pulls inner shaft 740 proximally relative to vessel protector device 700 to vertically collapse and radially expand basket 750. As seen in FIG. 7B, with the basket collapsed, shield 730 takes the shape shown in FIG. 7C, with the petals 735 covering the ostia to the artery (e.g., brachiocephalic artery 106) as desired while allowing an operative catheter to perform its intended function. That is, shield 730 expands into a shape that acts to filter blood passing through its wall to an artery. In addition, petals 735 may be formed large enough to cover more than one artery (e.g., brachiocephalic artery 106 and left common carotid artery 104).

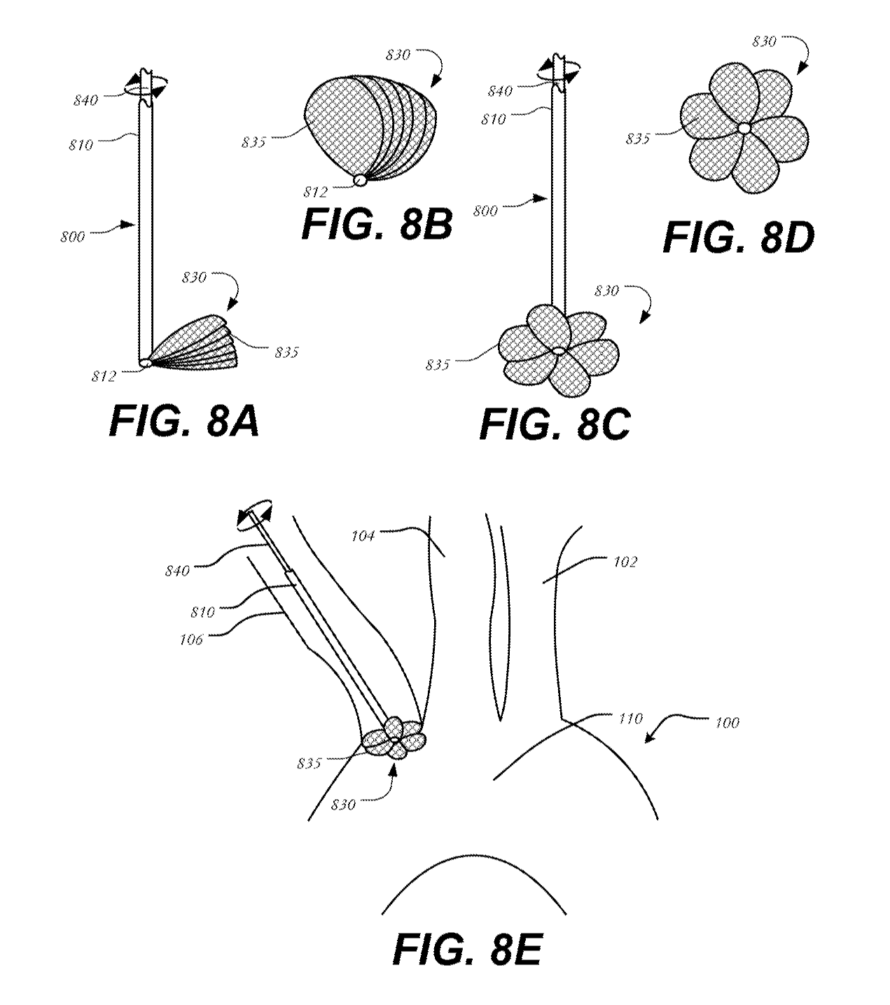

FIG. 8A is a side perspective view of vessel protector device 800 in accordance with another embodiment in a collapsed condition. As shown in this collapsed condition, vessel protector device 800 includes an elongated outer sheath 810 and inner shaft 840 disposed coaxially within outer sheath 810. Sheath 810 and shaft 840 are capable of rotation with respect to one another. Sheath 810 may be sized according to the vessel in which it will be used, or through which it will be deployed, and may be deployed within a delivery catheter such as delivery catheter.

Vessel protector device 800 further includes a plurality of blades 835 connected at their ends to sheath 810 and shaft 840 at pivot 812. Blades 835 may be oval, flat or curved or of other shapes. In the collapsed condition, blades 835 may be substantially overlapping one another as shown in FIGS. 8A and 8B. The blades 835 may be joined together such that rotation of shaft 840 causes blades 835 to spread out into the shape of a fan, blades 835 overlapping one another to form a shield. In one example, each blade 835 may be joined to an adjacent blade 835 at side edges such that when a first blade is rotated or pulled around a pivot, the next blade in the sequence follows it. In some examples, blades 835 may be joined via adhesive, a suture or fine wire. A first blade may be joined to the sheath 810 and the last blade joined to inner shaft 840. Thus, when sheath 810 is held in place and inner shaft 840 rotated, the blades may begin to spread out into the expanded fan-shaped configuration (shown in FIG. 8D). It will be understood that this example is provided by way of illustration and other methods of spreading out blades 835 may be used. For example, alternatively, blades 835 may be integrally formed to resemble a folding hand fan such that pulling on one end spreads the fan into an expanded configuration capable of acting as a shield.

Blades 835 may be formed from a woven, braided, or knitted material having openings of sufficient size to allow the passage of blood, but block the passage of particulates greater than a certain size. As such, the material of blades 835 acts as a filter. Each blade 835 may be formed of any of the materials described above with reference to body 235 and may be configured in the same manner as body 235 such as, for example, to include openings of varying sizes.

FIG. 8C is a side perspective view of the vessel protector device 800 of FIG. 8A in an expanded condition. Expansion of vessel protector device 800 as previously discussed may be accomplished by rotating shaft 840 relative to shaft 810. Blades 835 may spread out in an expanded fan-like configuration. As a result, blades 835 assume an expanded configuration to form shield 830 as shown in FIG. 8D. Shield 830 includes a plurality of partially overlapping blades 830 that collectively form a filtering barrier across a cross-section of an artery.

FIG. 8E is schematic illustration of the use of the vessel protector device of FIG. 8A in the aorta. As shown in this figure, vessel protector device 800 may be introduced toward aorta 100 in the collapsed configuration of FIG. 8A through brachiocephalic artery 106. Once protector device 800 is properly positioned, the user twists or otherwise rotates inner shaft 840 relative to sheath 810 to spread out the blades 835. As seen in FIG. 8E, with blades spread, shield 830 takes the shape shown in FIG. 8D, and covers the opening to an artery (e.g., brachiocephalic artery 106) as desired while allowing an operative catheter to perform its intended function. As with the petals of the previous embodiment, blades 835 may be formed large enough to cover additional arterial ostia.

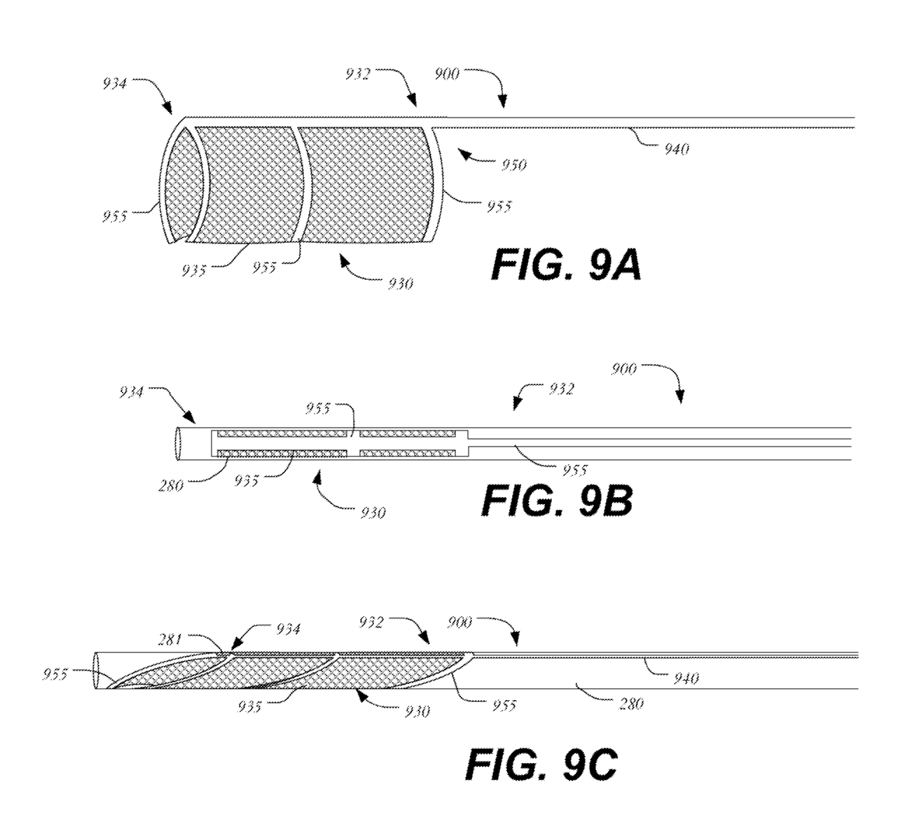

FIG. 9A is a side perspective view of vessel protector device 900 in accordance with another embodiment. Vessel protector device 900 includes elongated shaft 940 and shield 930 attached to the distal end of shaft 940 by a plurality of spaced-apart ribs 955. Shield 930 extends between leading end 934 and trailing end 932 and includes a plurality of panels 935 attached between adjacent ribs 955 on opposite sides of the device. As seen in FIG. 9A, ribs 955 are uniformly formed with shaft 940 to form frame 950. Ribs 955 may be shaped as curved portions or arches projecting from shaft 940. Frame 950, including shaft 940 and ribs 955, may be formed of any suitable metal or polymer. In one example, frame 950 may be formed from a biocompatible elastic, superelastic, elastomeric, or shape-memory material which returns to an initial undeformed shape upon release from a catheter. Alternatively, frame 950 which is not self-expanding may be formed from a biocompatible material which deforms plastically, and may employ additional snares or other devices to effect radial expansion.

A plurality of panels 935 formed of the filtering materials described above may be stretched between adjacent ribs to form a flat or curved shield 930. Each panel 935 may be formed of the same materials and include the same mesh arrangements described above with reference to FIGS. 2 and 3 and include a number of openings for filtering blood passing through the body.

FIG. 9B is a side perspective view of vessel protector device 900 of FIG. 9A in a first collapsed condition. Vessel protector device 900, including the frame 950 and shield 930, has been radially collapsed to reduce the profile of the device. Specifically, each rib 955 may be formed as flexible members so as to overlap a contralateral rib to facilitate radial collapse. The device may then be disposed within delivery catheter 280. Unsheathing frame 950 from delivery catheter 280 allows the frame to expand and deploy shield 930 as shown in FIG. 9A.

FIG. 9C is a side perspective view of vessel protector device 900 of FIG. 9A in an alternative collapsed configuration. In this collapsed configuration, the device may be collapsed in delivery catheter 280 having stop member 281. Stop member 281 may restrain shaft 240 near leading end 934, while ribs 955 are advanced forward of the stop member, thereby shearing the device as shown in FIG. 9C. The folded device 900 may be disposed within delivery catheter 280 and introduced into the site of filtration (e.g. the aortic arch) to protect one or more vessels. To release vessel protector device 900 from delivery catheter 280, the protector device may be rotated with respect to the delivery catheter to release shaft 240 from stop member 281.

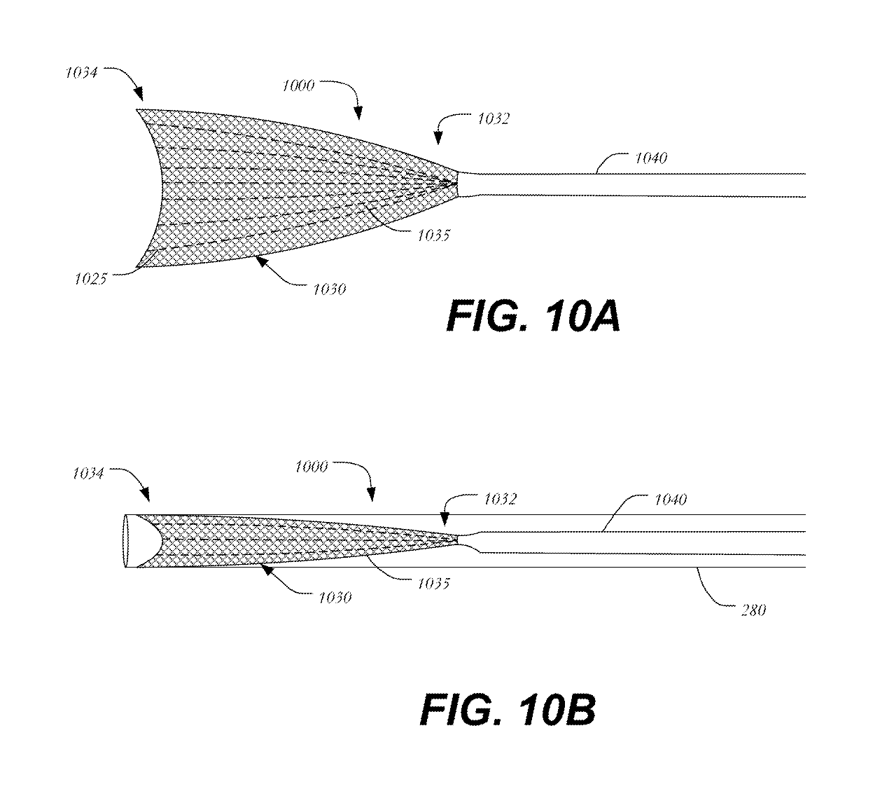

FIG. 10A is a side perspective view of vessel protector device 1000 in accordance with another embodiment. Protector device 1000 of FIG. 10A includes shield 1030 extending between leading end 1034 and trailing end 1032 and shaft 1040 attached to trailing end 1034 of shield 1030. Shield 1030 includes body 1035 formed of any of the filtering materials and in any of the configurations described above. In this embodiment, body 1035 is formed to have a curved or arced relaxed shape similar to the shape described above with reference to FIG. 5A. To aid in collapsing awning-shaped body 1035, the body may include a number of longitudinal pleats 1025. In this regard, body 1035 may be capable of folding upon itself along pleats 1025 in an accordion-like manner.

FIG. 10B illustrates vessel protector 1000 in a collapsed configuration and disposed within delivery catheter 280. As seen, pleats 1025 may facilitate body 1035 folding into a collapsed configuration having a smaller diameter for delivery to traverse the patient's vasculature. Specifically, body 1035 may collapse when a small diameter delivery catheter 280 sheathes it. Pleats 1025 may facilitate the collapse of body 1035. As described in previous embodiments, body 1035 may be formed of a shape-memory material such that exposing body 1035 from delivery catheter 280 allows body 1035 to expand to its relaxed configuration.

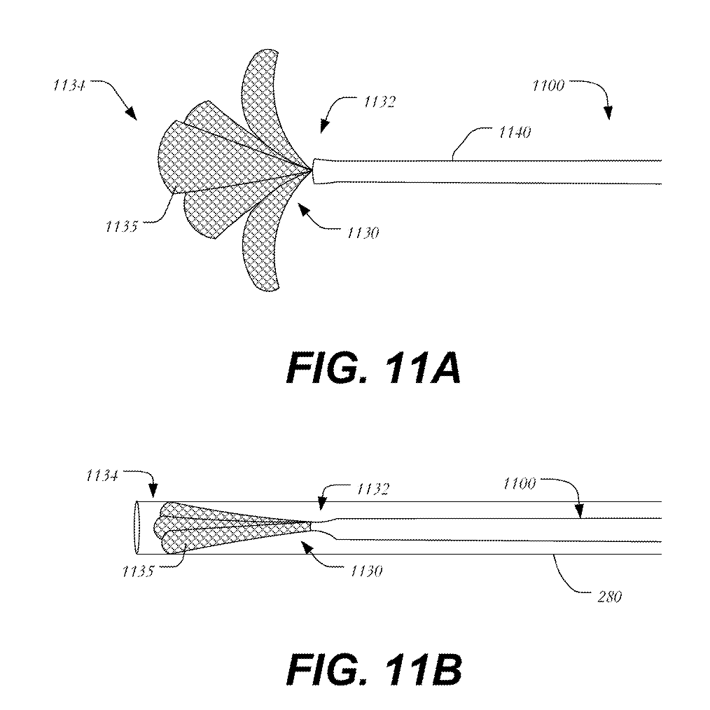

FIG. 11A is a side perspective view of a vessel protector device 1100 in accordance with another embodiment. Vessel protector device 1100 may include shaft 1140 similar to that of FIG. 10A and shield 1130 near leading end 1134. Shield 1130 includes a plurality of leaflets 1135 attached to shaft 1140 at trailing end 1132. Each leaflet 1135 may be formed as a mesh for filtering the blood and may be shaped as an elongated flower petal or other shape that is outwardly biased radially.

Leaflets 1135 are configured to expand from their bunched, collapsed state to the flower-shape shown in FIG. 11A. As seen in FIG. 11B, vessel protector device 1100 may be collapsed to fit within a delivery catheter 280 similarly to protector device 1000. When protector 1100 is unsheathed from delivery device 280, the individual leaflets 1135 expand radially outwardly to form a cross-section capable of blocking the ostia of an artery to filter blood through the filtering material of leaflets 1135. It will be understood that though FIGS. 11A and 11B illustrate a vessel protector device 1100 having five leaflets 1135, shield 1130 may include any number of leaflets 1135 including a single leaflets or multiple leaflets 1135 such as two, three, four, five, six, seven or more leaflets.

Although the devices, systems and methods herein have been described with reference to particular embodiments, it is to be understood that these embodiments are merely illustrative of the principles and applications of the present system and method. It is therefore to be understood that numerous modifications may be made to the illustrative embodiments and that other arrangements and combinations may be devised without departing from the spirit and scope of the present system and method as defined by the appended claims.

Any one or more of the following features can be combined with any of the embodiments described above. For example, the shield may be coupled to the inner rod at a leading end and to the outer sheath at a trailing end. The shield may be formed of a shape-memory material, and may move to the collapsed configuration from the expanded configuration when the outer sheath is pulled toward a proximal end relative to the inner rod. The filtering material may be at least one of a mesh, a braided material, a shape memory material or a nickel titanium alloy. The body may form a conical shape in its expanded configuration. The device may further include a pair of radiopaque marker bands coupled to the first and second ends of the shield.

The shield may also form a C-shaped configuration when deployed from the outer sheath. The body may be constructed of a single-layer tube that is folded over itself to form a double-layer tube that is collapsed to form a C-shaped configuration. Each of the plurality of shields may be formed as deformable petals coupled to the inner rod at a leading end and to the outer sheath at a trailing end, and moving the outer shield relative to the inner rod toward the leading end of the petals may flatten the petals from a basket-like collapsed configuration to a flower-like expanded configuration. Each of the petals may partially overlap with one another in the expanded configuration. Each of the plurality of shields may be formed as blades coupled to the inner rod, and rotating the inner rod relative to the outer shield may spread the blades into the expanded configuration.

In some other examples, a vessel protector includes a rod having a first end and a second end and at least one shield coupled to the first end of the rod, the at least one shield having a body formed from a filtering material. The at least one shield may be capable of collapsing to fit within a delivery catheter. The body of the at least one shield may have an expanded shape of an awning and a number of longitudinal pleats to aid in collapsing the body. At least one shield may include a plurality of leaflets formed of a shape-memory material that can be collapsed within a delivery catheter and returned to a radially expanded relaxed state when deployed from the delivery catheter.

Additionally, the method for protecting blood vessels may further include disposing the vessel protector device within a delivery catheter, introducing the delivery catheter into the body of the patient and deploying the vessel protector device from within the delivery catheter prior to positioning the vessel protector device adjacent an open end of at least one blood vessel.

It will be appreciated that the various dependent claims and the features set forth therein can be combined in different ways than presented in the initial claims. It will also be appreciated that the features described in connection with individual embodiments may be shared with others of the described embodiments.

1. A vessel protector for use with a pigtail catheter, comprising:
a pigtail catheter;
an outer sheath; and
a shield disposed within the outer sheath and having a body formed from a filtering material;
wherein the shield is capable of receiving the pigtail catheter.

2. The vessel protector according to claim 1, wherein the shield is formed of a shape-memory material, and wherein the shield moves to a collapsed configuration from an expanded configuration when the outer sheath is pushed distally over the shield.

3. The vessel protector according to claim 1, wherein the filtering material is at least one of a mesh, a braided material, a shape memory material or a nickel titanium alloy.

4. The vessel protector according to claim 1, wherein the pigtail catheter is capable of delivering contrast media.

5. The vessel protector according to claim 1, further comprising at least one radiopaque marker band coupled to the shield.

6. The vessel protector according to claim 1, further comprising an introducer catheter capable of housing at least a portion of the outer sheath.

7. The vessel protector according to claim 6, wherein the introducer catheter extends from a proximal end of the vessel protector to a location prior to a distal end of the vessel protector.

8. The vessel protector according to claim 1, wherein the shield forms a C-shaped configuration when deployed from the outer sheath.

9. The vessel protector according to claim 1, wherein the body is constructed of a single-layer tube that is folded over itself to form a double-layer tube that is collapsed to form a C-shaped configuration.

10. A vessel protector, comprising:
an outer sheath;
an inner shaft disposed within the outer sheath and moveable relative to the outer sheath; and
a plurality of shields coupled to the inner shaft, each of the plurality of shields having a body formed from a filtering material, the plurality of shields having a collapsed configuration and an expanded configuration;
wherein the plurality of shields are capable of alternating between the collapsed configuration and the expanded configuration by movement of the inner shaft relative to the outer sheath.

11. The vessel protector according to claim 10, wherein each of the plurality of shields are formed as deformable petals coupled to the inner shaft at a leading end and the outer sheath at a trailing end, and wherein moving the outer sheath relative to the inner shaft toward the leading end of the petals flattens the petals from a basket-like collapsed configuration to a flower-like expanded configuration.

12. The vessel protector according to claim 11, wherein each of the petals partially overlap with one another in the expanded configuration.

13. The vessel protector according to claim 10, wherein each of the plurality of shields are formed as blades coupled to the inner shaft, and wherein rotating the inner shaft relative to the outer sheath spreads the blades into the expanded configuration.

14. A vessel protector for use with a delivery catheter, comprising:
a frame including a shaft and a plurality of arched ribs connected to the shaft, the frame being formed of a shape-memory material that can be collapsed within the delivery catheter and returned to its expanded relaxed state when deployed from the delivery catheter; and
a plurality of shields disposed between the plurality of arched ribs, each of the plurality of shields having a body formed from a filtering material;
wherein the frame is capable of collapsing to fit within a delivery catheter.

15. A vessel protector, comprising:
a shaft having a first end and second end; and
at least one shield coupled to the first end of the shaft, the at least one shield having a body formed from a filtering material, the body having an expanded shape of an awning and a number of longitudinal pleats to aid in collapsing the body;
wherein the at least one shield is capable of collapsing to fit within a delivery catheter.

16. A vessel protector, comprising:
a shaft having a first end and second end; and
at least one shield coupled to the first end of the shaft, the at least one shield having a body formed from a filtering material, the at least one shield including a plurality of leaflets formed of a shape-memory material that can be collapsed within a delivery catheter and returned to a radially expanded relaxed state when deployed from the delivery catheter;
wherein the at least one shield is capable of collapsing to fit within a delivery catheter.

17. A method for protecting blood vessels during a medical procedure, comprising:
inserting a vessel protector device into a patient's body, the vessel protector device including an outer sheath, an inner shaft disposed within the outer sheath and moveable in a longitudinal direction relative to the outer sheath, and at least one shield coupled to the inner shaft at a first end of the shield and to outer sheath at a second end of the shield, each of the at least one shield having a body formed from a filtering material, the body having a collapsed configuration and an expanded configuration;
positioning the vessel protector device adjacent an open end of at least one blood vessel; and
moving the outer sheath relative to the inner shaft to place the body of the at least one shield in the expanded configuration to filter blood passing through the body into the at least one blood vessel.

18. The method according to claim 17, further comprising disposing the vessel protector device within a delivery catheter, introducing the delivery catheter into the body of the patient and deploying the vessel protector device from within the delivery catheter prior to positioning the vessel protector device adjacent an open end of at least one blood vessel.
